(19) United States
(12) Patent Application Publication
(10) Pub. No.: US 2024/0177843 A1
(43) Pub. Date: May 30, 2024

(54) ARTIFICIAL INTELLIGENCE BASED VIRTUAL ASSISTANT FOR HOSPITAL MANAGEMENT

(71) Applicant: Prince Sattam bin Abdulaziz University, AI-Kharj (SA)

(72) Inventors: Shtwai Alsubai, Al-Kharj (SA); Abdullah Alqahtani, Al-Kharj (SA); Abed Alanazi, Al-Kharj (SA); Ashit Kumar Dutta, Riyadh (SA)

(73) Assignee: Prince Sattam bin Abdulaziz University, AI-Kharj (SA)

(21) Appl. No.: 18/437,577

(22) Filed: Feb. 9, 2024

(51) Int. Cl. G16H 40/20; G06Q 40/08; G16H 10/60

(52) U.S. Cl. CPC G16H 40/20; G06Q 40/08; G16H 10/60

(57) ABSTRACT

The “Artificial Intelligence-Based Virtual Assistant for Hospital Management” is a transformative system revolutionizing healthcare administration. Employing adaptive machine learning algorithms, the virtual assistant dynamically optimizes appointment scheduling, considering historical data, real-time factors, and patient preferences. Natural language processing ensures seamless communication with patients and facilitates dynamic rescheduling. An intelligent patient record management module securely organizes unstructured data, enhancing access for healthcare professionals. The system automates billing and insurance processing with AI oversight, identifying discrepancies and ensuring regulatory compliance. Real-time resource allocation dynamics optimize bed utilization based on predictive analytics. An emergency response coordination module interprets and prioritizes messages, initiating protocols during critical events. The system offers an adaptable user interface with real-time analytics. This invention marks a significant leap in healthcare technology, fostering efficiency, accuracy, and patient-centricity in hospital management.

### FIELD OF THE INVENTION

The present invention relates to the field of healthcare technology and, more specifically, pertains to an Artificial Intelligence based virtual assistant for hospital management. The invention encompasses the integration of advanced machine learning algorithms, natural language processing, and data analytics to optimize and automate various administrative and operational aspects within a healthcare facility.

### BACKGROUND OF THE INVENTION

The healthcare industry plays a pivotal role in society, continuously evolving to meet the growing demands of an expanding and diverse patient population. Amidst these advancements, the integration of technology has emerged as a critical factor in addressing the challenges faced by healthcare institutions, particularly in the realm of hospital management.

Traditionally, hospital management has been a complex and multifaceted task, involving the coordination of numerous activities, including but not limited to appointment scheduling, patient record management, billing, and resource allocation. The increasing volume of patients, coupled with the intricacies of healthcare systems, has underscored the need for innovative solutions that can enhance operational efficiency while ensuring optimal patient outcomes.

In recent years, Artificial Intelligence (AI) has proven to be a transformative force in various industries, and healthcare is no exception. AI technologies, such as machine learning and natural language processing, have demonstrated their potential to revolutionize traditional healthcare practices. The application of AI in hospital management has the capacity to streamline processes, reduce errors, and ultimately contribute to a more patient-centric and cost-effective healthcare system.

The proposed invention recognizes the challenges faced by healthcare institutions and seeks to address them through the development of an AI-based virtual assistant tailored for hospital management. This virtual assistant is envisioned as an intelligent system capable of learning, adapting, and optimizing a myriad of administrative and operational tasks, thereby alleviating the burdens associated with manual processes.

By leveraging the power of AI, the virtual assistant aims to bring about improvements in appointment scheduling, patient record management, billing and insurance processing, resource allocation, and emergency response coordination. The goal is to create a seamless and efficient environment within healthcare institutions, fostering a more focused and personalized approach to patient care.

The ever-growing volume of patient data, coupled with the need for timely and accurate decision-making, necessitates a paradigm shift in hospital management. The invention outlined in this application represents a significant step towards harnessing the capabilities of AI to enhance the overall efficiency, accuracy, and responsiveness of healthcare systems, ultimately contributing to the advancement of patient care and the optimization of healthcare resources.

### SUMMARY OF THE INVENTION

The “Artificial Intelligence-Based Virtual Assistant for Hospital Management” is a groundbreaking solution designed to revolutionize the operational landscape of healthcare institutions. Leveraging cutting-edge technologies such as Artificial Intelligence, machine learning, and natural language processing, the virtual assistant serves as an intelligent and adaptive system dedicated to enhancing the efficiency and effectiveness of hospital management processes.

### Key Features:

### Appointment Scheduling Optimization:

The virtual assistant employs AI algorithms to intelligently optimize appointment scheduling, taking into account factors such as physician availability, patient preferences, and urgency.

### Patient Record Management:

Utilizing advanced data analytics and natural language processing, the virtual assistant ensures secure and efficient management of patient records, enabling quick and accurate access to crucial healthcare information.

### Billing and Insurance Processing Automation:

The system streamlines billing processes and automates insurance claim submissions, reducing errors, expediting reimbursement cycles, and minimizing administrative overhead.

### Resource Allocation Efficiency:

Real-time data analytics are utilized to optimize resource allocation within the hospital, ensuring optimal bed utilization, minimizing wait times, and enhancing overall operational efficiency.

### Emergency Response Coordination:

The virtual assistant includes an emergency response coordination module, enabling swift and effective communication and coordination during critical situations, ultimately improving crisis management within the healthcare facility.

### Advantages:

Operational Efficiency: The virtual assistant enhances the efficiency of hospital management processes, reducing manual workload and allowing healthcare professionals to focus on patient care.
Patient-Centric Approach: By automating administrative tasks, the virtual assistant contributes to a more patient-centric healthcare environment, ensuring timely and personalized care.
Error Reduction: Automation minimizes human errors in tasks such as billing and record management, contributing to the overall accuracy and reliability of healthcare processes.
Resource Optimization: Through real-time analytics, the system aids in optimal resource allocation, contributing to improved bed utilization and resource efficiency.

In summary, the “Artificial Intelligence-Based Virtual Assistant for Hospital Management” represents a transformative leap towards a more intelligent, responsive, and patient-focused healthcare management system. By seamlessly integrating AI technologies into the daily operations of healthcare institutions, this invention aims to elevate the standard of care delivery and contribute to the overall advancement of the healthcare industry.

### BRIEF DESCRIPTION OF THE DRAWINGS

FIG. 1 is a diagram of an artificial intelligence based virtual assistant for hospital management.

### DETAILED DESCRIPTION OF THE INVENTION

The “Artificial Intelligence-Based Virtual Assistant for Hospital Management” introduces a paradigm shift in healthcare technology, embodying a novel and pioneering approach to address the intricacies of hospital management. The invention discloses unique amalgamation of Artificial Intelligence (AI), machine learning, and natural language processing, creating an integrated system that redefines traditional practices in healthcare administration.

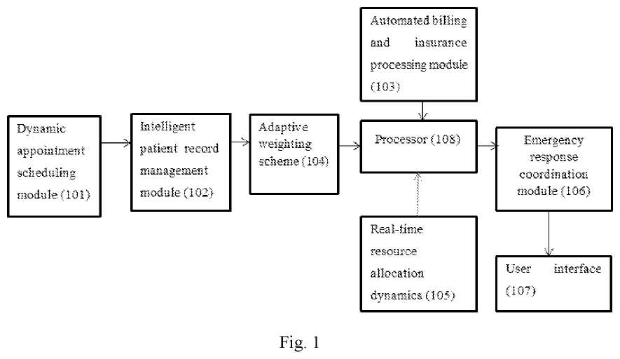

FIG. 1 is a diagram of an artificial intelligence based virtual assistant for hospital management. The artificial intelligence based virtual assistant for hospital management comprising of a dynamic appointment scheduling module (101), an intelligent patient record management module (102), an automated billing and insurance processing module (103), an adaptive weighting scheme (104), a real-time resource allocation dynamics (105), an emergency response coordination module (106), user interface (107) and a processor (108).

This innovative virtual assistant brings forth dynamic appointment scheduling optimization, utilizing adaptive machine learning algorithms to consider historical data, real-time factors, and patient preferences. Unlike conventional approaches, it dynamically adjusts schedules to maximize efficiency and adapt to changing circumstances.

In the realm of patient record management, the system showcases a groundbreaking use of natural language processing, enabling the intelligent extraction and categorization of unstructured patient data. This capability provides healthcare professionals with unprecedented quick and accurate access to relevant information, surpassing the capabilities of existing record management systems.

The automated billing and insurance processing system break new ground by not only streamlining processes but also introducing AI oversight to identify billing discrepancies and ensure compliance with regulatory requirements. This represents a departure from traditional billing systems, introducing a higher level of accuracy and efficiency.

Real-time resource allocation dynamics present a novel approach, with the system dynamically adjusting bed utilization and resource allocation based on predictive analytics. This adaptive functionality goes beyond the capabilities of current hospital management systems, optimizing resources in real-time for enhanced efficiency.

The inclusion of a dedicated emergency response coordination module, driven by AI, represents a novel approach to crisis management within healthcare settings. The system's ability to interpret and prioritize emergency messages, initiate communication protocols, and provide real-time updates establishes a new standard for responsiveness during critical events, setting it apart from existing emergency response systems.

In essence, the “Artificial Intelligence-Based Virtual Assistant for Hospital Management” is not merely an incremental improvement but a transformative leap forward. By seamlessly integrating AI and related technologies, this invention redefines the landscape of hospital management, setting a new standard for intelligent, adaptive, and patient-centric healthcare systems.

The “Artificial Intelligence-Based Virtual Assistant for Hospital Management” is an innovative system designed to revolutionize the way healthcare institutions manage their operations. This detailed description provides an in-depth exploration of the technological aspects, algorithms utilized, and the functionality of the virtual assistant across various hospital management processes.

### 1. Appointment Scheduling Optimization:

The virtual assistant incorporates advanced machine learning algorithms to optimize the appointment scheduling process. By analyzing historical data, patient preferences, and physician availability, the system intelligently suggests and adjusts appointment times to maximize efficiency. Natural language processing capabilities enable seamless communication with patients, allowing for the dynamic rescheduling of appointments based on changing circumstances.

### 2. Patient Record Management:

Utilizing state-of-the-art data analytics and natural language processing, the virtual assistant ensures the secure and efficient management of patient records. The system extracts relevant information from unstructured data, such as clinical notes and reports, and organizes it into a structured database. This facilitates quick and accurate retrieval of patient information by authorized healthcare professionals, contributing to improved diagnostic accuracy and timely decision-making.

### 3. Billing and Insurance Processing Automation:

The virtual assistant automates billing processes and insurance claim submissions, minimizing errors and accelerating reimbursement cycles. Machine learning algorithms analyze billing patterns, identify discrepancies, and ensure compliance with regulatory requirements. The system utilizes natural language processing to interpret insurance documents, facilitating accurate claim submissions and reducing the administrative burden on hospital staff.

### 4. Resource Allocation Efficiency:

Real-time data analytics play a pivotal role in optimizing resource allocation within the hospital. The virtual assistant analyzes patient data, bed availability, and resource utilization patterns to dynamically allocate resources, ensuring optimal bed utilization and minimizing patient wait times. Machine learning models continuously adapt to changing conditions, allowing for proactive resource allocation based on predicted demand.

### 5. Emergency Response Coordination:

The virtual assistant includes a dedicated module for emergency response coordination. In critical situations, the system automatically triggers alerts to relevant departments, initiates communication protocols, and provides real-time updates to healthcare professionals. Natural language processing enables the system to interpret and prioritize emergency messages, ensuring a swift and coordinated response to critical events.

### Technical Architecture:

The virtual assistant is built on a robust and scalable architecture, incorporating cloud computing for seamless data storage and processing. Machine learning models are trained on vast datasets, continuously learning and adapting to evolving hospital dynamics. Application programming interfaces (APIs) enable integration with existing hospital information systems, ensuring interoperability and a smooth transition into existing workflows.

### User Interface:

The virtual assistant features an intuitive user interface accessible to healthcare professionals, administrators, and, where applicable, patients. The interface provides a holistic view of scheduled appointments, patient records, billing status, and resource utilization. Customizable dashboards and real-time analytics empower users with actionable insights for informed decision-making.

In conclusion, the “Artificial Intelligence-Based Virtual Assistant for Hospital Management” represents a comprehensive and intelligent solution for transforming the operational landscape of healthcare institutions. By leveraging advanced technologies, the virtual assistant aims to enhance efficiency, reduce errors, and ultimately contribute to the delivery of high-quality, patient-centered care.

In an embodiment of the present invention, the comprehensive Artificial Intelligence-Based Virtual Assistant System for Hospital Management, comprising: A dynamic appointment scheduling module utilizing adaptive machine learning algorithms to optimize scheduling based on historical data, real-time factors, and patient preferences;

An intelligent patient record management module incorporating advanced natural language processing for the extraction, categorization, and secure management of unstructured patient data, facilitating quick and accurate access for healthcare professionals; An automated billing and insurance processing module featuring AI oversight to identify discrepancies, ensure regulatory compliance, and streamline the billing and reimbursement processes; Real-time resource allocation dynamics for optimizing bed utilization and resource allocation within the hospital based on predictive analytics; An emergency response coordination module, utilizing AI, to interpret and prioritize emergency messages, initiate communication protocols, and provide real-time updates during critical events for efficient crisis management; and

An adaptable user interface offering healthcare professionals, administrators, and patients a customizable dashboard with real-time analytics for informed decision-making.

What is claimed is:

1. A comprehensive Artificial Intelligence-Based Virtual Assistant System for Hospital Management, comprising:
A dynamic appointment scheduling module utilizing adaptive machine learning algorithms to optimize scheduling based on historical data, real-time factors, and patient preferences;
An intelligent patient record management module incorporating advanced natural language processing for the extraction, categorization, and secure management of unstructured patient data, facilitating quick and accurate access for healthcare professionals; and
An automated billing and insurance processing module featuring AI oversight to identify discrepancies, ensure regulatory compliance, and streamline the billing and reimbursement processes;
Real-time resource allocation dynamics for optimizing bed utilization and resource allocation within the hospital based on predictive analytics;
An emergency response coordination module, utilizing AI, to interpret and prioritize emergency messages, initiate communication protocols, and provide real-time updates during critical events for efficient crisis management; and
An adaptable user interface offering healthcare professionals, administrators, and patients a customizable dashboard with real-time analytics for informed decision-making.

2. The system of claim 1, wherein the dynamic appointment scheduling module further employs natural language processing for seamless communication with patients and facilitates dynamic rescheduling based on changing circumstances.

3. The system of claim 1, wherein the automated billing and insurance processing module utilizes natural language processing to interpret insurance documents, ensuring accurate claim submissions and reducing the administrative burden on hospital staff.

4. The system of claim 1, wherein the user interface provides a holistic view of scheduled appointments, patient records, billing status, and resource utilization, empowering users with actionable insights for informed decision-making.

5. The system of claim 1, wherein the virtual assistant is built on a scalable architecture, leveraging cloud computing for seamless data storage and processing, ensuring scalability, and interoperability with existing hospital information systems through application programming interfaces.

6. The system of claim 1, further comprising machine learning models continuously adapting to changing conditions, allowing for proactive resource allocation based on predicted demand for enhanced operational efficiency.

7. The system of claim 1, wherein the emergency response coordination module includes predefined communication protocols and automated triggers for swift and coordinated responses during critical events, ensuring a heightened level of responsiveness.

8. A method for hospital management utilizing an Artificial Intelligence-Based Virtual Assistant, comprising the steps of:
dynamically optimizing appointment schedules using adaptive machine learning algorithms that consider historical data, real-time factors, and patient preferences;
employing natural language processing for seamless communication with patients and dynamically rescheduling appointments based on changing circumstances;
extracting, categorizing, and securely managing unstructured patient data through an intelligent patient record management module;
streamlining billing and insurance processing with AI oversight to identify discrepancies and ensure regulatory compliance;
dynamically adjusting resource allocation, including bed utilization, based on real-time data analytics and predictive analytics; and
coordinating emergency responses through an AI-driven module that interprets and prioritizes emergency messages, initiates communication protocols, and provides real-time updates during critical events.

9. The method of claim 8, further comprising the step of: Providing an adaptable user interface presenting healthcare professionals, administrators, and patients with a customizable dashboard displaying real-time analytics for informed decision-making.

10. The method of claim 8, further comprising the step of: Utilizing natural language processing in the automated billing and insurance processing step to interpret insurance documents, facilitating accurate claim submissions and reducing the administrative burden on hospital staff.

11. The method of claim 8, further comprising the step of: Incorporating machine learning models that continuously adapt to changing conditions, allowing for proactive resource allocation based on predicted demand for enhanced operational efficiency.

12. The method of claim 8, further comprising the step of: Employing advanced data analytics and natural language processing to continuously monitor and analyze patient records, facilitating predictive diagnostics and personalized treatment plans.

13. The method of claim 8, additionally comprising the step of: Implementing proactive data security measures within the intelligent patient record management module, including encryption and access controls, ensuring the confidentiality and integrity of patient information.
